(19) United States
(12) Patent Application Publication
(10) Pub. No.: US 2024/0268715 A1
(43) Pub. Date: Aug. 15, 2024

(54) GLUCOSE LEVEL CHANGE DETECTION IN EYES USING POLARIZED LIGHT

(71) Applicant: Meta Platforms Technologies, LLC, Menlo Park, CA (US)

(72) Inventors: Mantas ZURAUSKAS, Redmond, WA (US); Jiamin Zheng, Mountain View, CA (US)

(73) Assignee: Meta Platforms Technologies, LLC, Menlo Park, CA (US)

(21) Appl. No.: 18/413,578

(22) Filed: Jan. 16, 2024

Related U.S. Application Data
(60) Provisional application No. 63/445,924, filed on Feb. 15, 2023.

(51) Int. Cl. A61B 5/145; A61B 5/00; A61B 5/1455; G02B 27/00; G02B 27/01

(52) U.S. Cl. CPC A61B 5/14532; A61B 5/14558; A61B 5/6803; G02B 27/0093; G02B 27/0172; A61B 2560/0406; A61B 2562/0233

(57) ABSTRACT

According to examples, an apparatus may include a processor and a memory on which is stored machine-readable instructions that when executed by the processor, cause the processor to access a detected polarization state of light reflected from an individual's eye, identify conditions associated with a detection of the polarization state of the light reflected from the individual's eye, determine whether the identified conditions match certain conditions within predefined difference levels, and determine a variance in the detected polarization state of the light from a previously detected polarization state based on a determination that the identified conditions match the certain conditions within the predefined difference levels, in which a change in a glucose level of the individual is to be determined based on the determined variance.

### TECHNICAL FIELD

This patent application relates generally to head-mounted devices (HMDs). Particularly, this patent application relates to HMDs having components, such as eye tracking systems, to measure glucose levels in the eyes of users of the HMDs. This patent application also relates to determining changes in users' glucose levels when conditions associated with the measurement of a glucose level are within predefined difference levels of conditions associated with a previous measurement of a glucose level.

### BACKGROUND

With recent advances in technology, prevalence and proliferation of content creation and delivery have increased greatly in recent years. In particular, interactive content such as virtual reality (VR) content, augmented reality (AR) content, mixed reality (MR) content, and content within and associated with a real and/or virtual environment (e.g., a “metaverse”) has become appealing to consumers.

Wearable devices, such as a wearable eyewear, wearable headsets, head-mountable devices, and smartglasses, have gained in popularity as forms of wearable systems. In some examples, such as when the wearable devices are eyeglasses or smartglasses, the wearable devices may include transparent or tinted lenses. In some examples, the wearable devices may employ imaging components to capture image content, such as photographs and videos. In some examples, such as when the wearable devices are head-mountable devices or smartglasses, the wearable devices may employ a first projector and a second projector to direct light associated with a first image and a second image, respectively, through one or more intermediary optical components at each respective lens, to generate “binocular” vision for viewing by a user.

### BRIEF DESCRIPTION OF DRAWINGS

Features of the present disclosure are illustrated by way of example and not limited in the following figures, in which like numerals indicate like elements. One skilled in the art will readily recognize from the following that alternative examples of the structures and methods illustrated in the figures can be employed without departing from the principles described herein.

FIG. 1 illustrates a block diagram of an apparatus that may determine a variance in a detected polarization state of light over a previously detected polarization state of light reflected from an individual's eye, according to an example.

FIGS. 2A and 2B, respectively, illustrate block diagrams of an eye tracking system that may be employed to detect a polarization state of light reflected from an individual's eye, according to examples.

FIG. 3 illustrates a top view of a head-mounted device in which either of the eye tracking systems depicted in FIGS. 2A, 2B may be incorporated, according to an example.

FIG. 4 illustrates a perspective view of a head-mounted device, such as a near-eye display device, that may include either of the eye tracking systems depicted in FIGS. 2A, 2B, according to an example.

FIG. 5 illustrates a flow diagram of a method for a variance in a detected polarization state of light over a previously detected polarization state of light reflected from an individual's eye, according to an example.

### DETAILED DESCRIPTION

For simplicity and illustrative purposes, the present application is described by referring mainly to examples thereof. In the following description, numerous specific details are set forth in order to provide a thorough understanding of the present application. It will be readily apparent, however, that the present application may be practiced without limitation to these specific details. In other instances, some methods and structures readily understood by one of ordinary skill in the art have not been described in detail so as not to unnecessarily obscure the present application. As used herein, the terms “a” and “an” are intended to denote at least one of a particular element, the term “includes” means includes but not limited to, the term “including” means including but not limited to, and the term “based on” means based at least in part on.

Blood glucose sensing is important for millions of people and particularly those who are diagnosed with diabetes. Currently, approximately 8.7% of the US population is diagnosed with diabetes and more than 1 in three US adults have pre-diabetes. Blood glucose sensing may be important for some diabetics because blood glucose levels that are too high or too low may lead to certain harmful medical conditions. If an individual knows that their blood glucose level is too high or too low, e.g., outside of a normal blood glucose level range, the individual may know to eat some food and/or take medication to bring their blood glucose levels to within or close to the normal blood glucose level range.

Blood glucose measurement devices typically require invasive measurements, such as the use of lancets to draw an individual's blood and test strips and a blood glucose meter to measure their blood glucose level. Other blood glucose measurement devices include continuously wearbable specialized measurement devices, such as continuous glucose monitoring systems, which include patches with sub-dermal needles that are inserted into a user's arm. Still other blood glucose measurement devices include contact lenses that may measure blood glucose levels in a user's tears. Known blood glucose measurement devices may thus be disruptive to their users, and in some instances, painful to the users.

Disclosed herein are apparatuses, methods, and computer-readable media for determining variances in detected polarization states of light over previously detected polarization states of light reflected from an individual's eye. Particularly, an illumination source may emit light at a known polarization state (e.g., angle) into the individual's eye. The polarization state of the light may change due to various conditions associated with the eye, including the glucose level in the eye. As a result, the light reflected from the eye may have a different polarization state than the light that is propagated through the eye and the difference level may be affected by the glucose level in the eye. For instance, the polarization state (rotation angle) difference may be greater for greater levels of glucose in the eye. Additionally, the glucose level in the eye may correlate to the blood glucose level in the individual's body.

According to examples, the apparatus, which may be a head-mounted device, may include an eye tracking system that may track various conditions of the individual's eye. For instance, the eye tracking system may track various conditions of the individual's eye that may affect the level of change in the polarization state. The various conditions may include the glucose level (e.g., the number of glucose molecules present in the eye), a pupil dilation level, an accommodation distance, a gaze direction, an illumination level caused by environmental lighting, and/or the like.

The eye tracking system may include the illumination source and a polarization sensitive detector. The polarization sensitive detector may detect the polarization state of the light reflected from the eye as well as the other conditions that may affect the polarization state of the reflected light. For instance, the polarization sensitive detector may detect the direction in which an individual is looking, the pupil dilation, etc.

According to examples, the conditions of the individual's eye present when the polarization state of the light reflected from the individual's eye was detected may be compared against the conditions of the individual's eye present during the detection of a previous polarization state of the reflected light. In instances in which the conditions from the different times differ significantly with respect to each other, e.g., beyond a predefined difference level, the polarization states detected at the different times may not be used to determine a change in the glucose level in the individual's eye. The polarization states may not be used because the differences in conditions may be a cause of differences in the polarization states and thus, the glucose level may not accurately be determined from the polarization state of the reflected light. However, when the conditions are similar to or match each other, a difference in the detected polarization state from the previously detected polarization state may be used to determine the glucose level in the eye. In other words, when the conditions are similar to or match each other, it may be likely that the glucose level is the cause of the change in the polarization state of the reflected light from the polarization state of the light emitted by the illumination source.

Through implementation of the features of the present disclosure, changes in a user's blood glucose level may be detected over time in a non-invasive and pain-free manner, e.g., without requiring that the user's blood be drawn. The user's blood glucose level may be correlated to the glucose level in the user's eye and thus, the user's blood glucose level may be determined through a determination of the glucose level in the user's eye. As discussed herein, a head-mounted device may include an eye tracking system that may detect changes in the polarization states of the light reflected from a user's eye multiple times, e.g., at certain intervals of time. In some instances, the eye tracking system may detect the changes as a background operation, e.g., without informing the user of this process. As a result, changes in a user's blood glucose level may be detected in a manner that may not require user instructions.

Additionally, by using polarization states that were detected under similar conditions as disclosed herein to determine whether a glucose level has changed, changes in glucose levels may be determined with greater accuracy. In other words, the number of false indications that the glucose level has changed may be reduced or minimized.

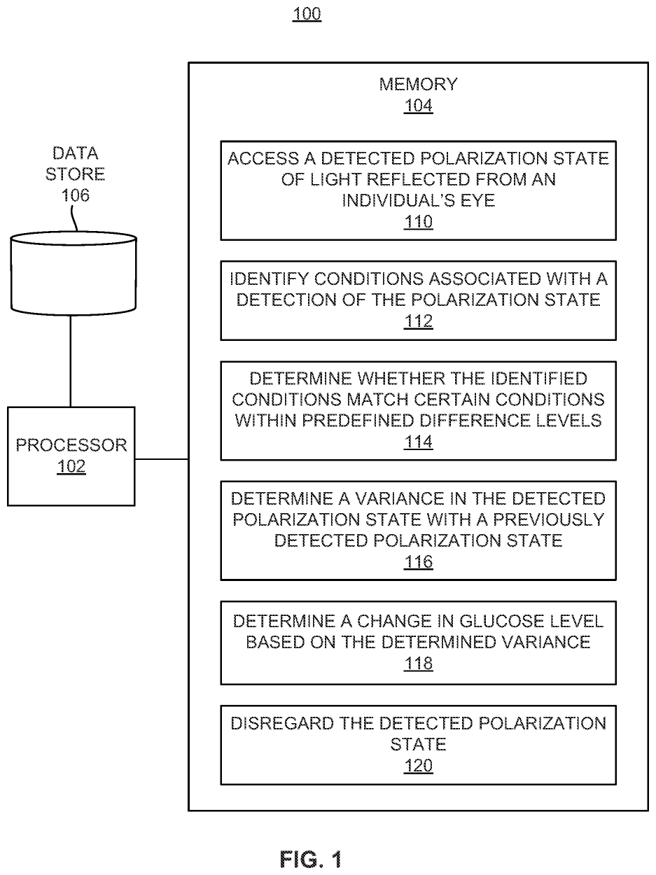

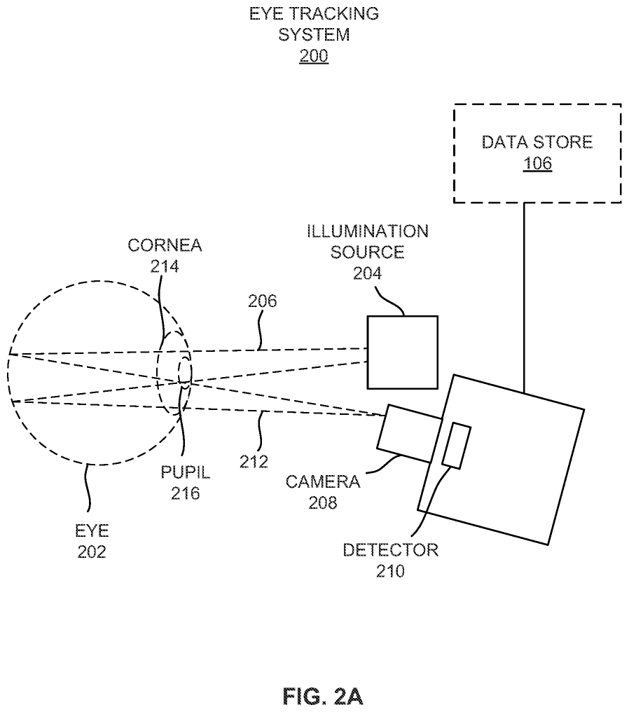

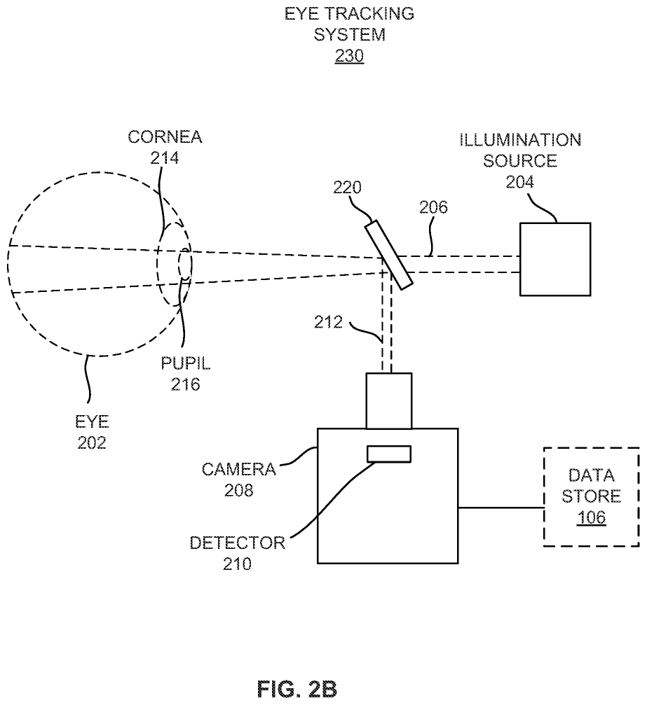

Reference is first made to FIGS. 1, 2A, and 2B. FIG. 1 illustrates a block diagram of an apparatus 100 that may determine a variance in a detected polarization state of light over a previously detected polarization state of light reflected from an individual's eye, according to an example. FIGS. 2A and 2B, respectively, illustrate block diagrams of eye tracking systems 200, 230 that may be employed to detect a polarization state of light reflected from an individual's eye 202, according to examples.

In some examples, the apparatus 100 may be a computing device, such as a smartphone, a laptop computer, a smartwatch, a tablet computer, a server, or the like. In these examples, the apparatus 100 may be in communication with a head-mounted device that may include the eye tracking system 200, 230 as discussed herein. In some examples, the apparatus 100 may be a head-mounted device that may include the eye tracking system 200, 230 as discussed herein.

In any of these examples, the apparatus 100 may include a processor 102 that may control some or all of the operations of the apparatus 100. The apparatus 100 may also include a memory 104 on which instructions that the processor 102 may access and/or execute are stored as discussed herein. In addition, the processor 102 may include a data store 106 on which the processor 102 may store various information as also discussed herein. The processor 102 may be a semiconductor-based microprocessor, a central processing unit (CPU), an application specific integrated circuit (ASIC), a field-programmable gate array (FPGA), and/or other hardware device. The memory 104, which may also be termed a computer readable medium, may, for example, a Random Access memory (RAM), an Electrically Erasable Programmable Read-Only Memory (EEPROM), a storage device, or the like. In some examples, the memory 104 is a non-transitory computer readable storage medium, where the term “non-transitory” does not encompass transitory propagating signals. In any regard, the memory 104 may have stored thereon machine-readable instructions that the processor 102 may execute. The data store 106 may also be a Random Access memory (RAM), an Electrically Erasable Programmable Read-Only Memory (EEPROM), a storage device, or the like.

Although the apparatus 100 is depicted as having a single processor 102, it should be understood that the apparatus 100 may include additional processors and/or cores without departing from a scope of the apparatus 100. In this regard, references to a single processor 102 as well as to a single memory 104 may be understood to additionally or alternatively pertain to multiple processors 102 and/or multiple memories 104. In addition, or alternatively, the processor 102 and the memory 104 may be integrated into a single component, e.g., an integrated circuit on which both the processor 102 and the memory 104 may be provided. In addition, or alternatively, the operations described herein as being performed by the processor 102 are distributed across multiple apparatuses 100 and/or multiple processors 102.

The memory 104 may have stored thereon machine-readable instructions 110-120 that the processor 102 may execute. Although the instructions 110-120 are described herein as being stored on the memory 104 and thus include a set of machine-readable instructions, the apparatus 100 may include hardware logic blocks that may perform functions similar to the instructions 110-120. For instance, the processor 102 may include hardware components that may execute the instructions 110-120. In other examples, the apparatus 100 may include a combination of instructions and hardware logic blocks to implement or execute functions corresponding to the instructions 110-120. In any of these examples, the processor 102 may implement the hardware logic blocks and/or execute the instructions 110-120. As discussed herein, the apparatus 100 may also include additional instructions and/or hardware logic blocks such that the processor 102 may execute operations in addition to or in place of those discussed herein.

The processor 102 may execute the instructions 110 to access a detected polarization state of light reflected from an individual's eye 202. For instance, and with reference to FIGS. 2A and 2B, the processor 102 may access the polarization state of light collected by an eye tracking system 200. As shown in FIGS. 2A and 2B, the eye tracking system 200 may include an illumination source 204 that may project light 206 into the eye 202, e.g., through the cornea 214 of the eye 202, at a certain polarization state, e.g., a linear polarization state. The polarized light 206 may be a collimated beam of light directed towards the individual's iris or retina (e.g., vasculature in the retina). In some instances, the polarized light 206 may be directed towards the individual's eye 202 via waveguides, lenses, prisms, mirrors, and/or the like, such that, for instance, the illumination source 204 may be positioned away from a direct line of sight of the eye 202.

The illumination source 204 may be any suitable type of illumination device that may output linearly polarized light, circularly polarized light converted to linearly polarized light, unpolarized light used with a polarizer, or the like. For instance, the illumination source 204, which may output unpolarized light, may include a polarizing filter positioned in front of the illumination source 204 such that light directed through the polarizing filter may be polarized light 206. For example, the polarizing filter may include a filter with a preferential plane of transmission to linearly polarize the light. In various examples disclosed herein, the illumination source 204 may include a polarizer that may utilize one or more principles of light transmission, reflection, scattering, constructive interference, and/or destructive interference.

According to examples, the illumination source 204 may output light at any suitable wavelength that may not be harmful to the human eye. For example, the illumination source 204 may output light at a wavelength that is in the range from about 400 nm to about 1500 nm. In some examples, the wavelength of light outputted by the illumination source 204 may be variable and/or the illumination source 204 may concurrently output light at multiple wavelengths. In some examples, the illumination source 204 may include a laser diode, e.g., a vertical-cavity surface-emitting laser (VCSEL), while in other examples, the illumination source 204 may include another type of illumination device such as a light emitting diode (LED), a narrowband LED, or the like. In some examples, the eye tracking system 200 may include multiple illumination sources 204 that may each output light at a different wavelength with respect to each other.

As shown in FIG. 2A, a portion of the projected polarized light 206 may be reflected, scattered and/or diffracted by various anatomical features and/or conditions of the individual's eye 202. The polarization state, e.g., the angle of polarization or the rotation angle, of the projected polarized light 206 may be rotated by some of the anatomical features of the eye 202. For instance, the type of the tissue in the eye 202, which may be approximately the same across the iris in a normal eye, a topology of the iris, corneal birefringence, and the glucose molecules in the aqueous humor. In other words, conditions associated with the eye 202 may affect the polarization state of the polarized light 206. The conditions may include a pupil 216 dilation level of the individual's eye 202, accommodation distance of the individual's eye 202, gaze direction of the individual's eye 202, and an illumination level, e.g., an ambient illumination level.

As discussed herein, the processor 102 may determine when the conditions, other than glucose levels, are the same or are within predefined difference levels with each other when multiple polarization state measurements are made. As a result, when the conditions are the same or are within predefined difference levels with each other and the polarization state has changed between two or more measurements, the processor 102 may determine that the polarization state has changed likely due to a change in the glucose levels in the eye 202. Generally speaking, glucose exists in the aqueous humor in both the anterior and posterior chambers of the eye 202. Glucose molecules are chiral molecules that may cause the polarization state, e.g., angle, of linearly polarized light (e.g., the plane of polarization) to rotate. The amount of the polarization angle rotation may be related to the glucose concentration, for instance, there may be a proportional relationship between the amount of the polarization angle rotation and the glucose concentration. For instance, the greater the polarization angle rotation, the greater the glucose concentration and the lesser the polarization angle rotation, the lesser the glucose concentration.

As shown in FIG. 2A, the eye tracking system 200 may include a camera 208 and a detector 210. The camera 208 may include various optical components, e.g., lenses, filters, etc., to capture polarized light 212 reflected from the eye 202. The captured polarized light 212 may be directed to the detector 210, which may analyze the polarized light 212 to determine the polarization angle rotation of the polarized light 212. The detector 210 may include electronics configured to determine the angle of rotation of the polarized light 212.

The detector 210 may include a polarizer filter, which may be positioned such that when the reflected polarized light 212 has rotated, the polarizing filter blocks at least a portion of the polarized light 212 because the polarization of the light is not aligned with the plane of transmission of the polarizing filter. In some examples, the polarizing filter may be rotated such that the plane of transmission of the polarizing filter may be aligned with the polarization of the reflected polarized light 212 and transmission may be increased or maximized. The polarization angle rotation of the reflected polarized light 212 may be determined by the amount of rotation that causes the most reflected light 212 to transmit through the polarizing filter. In some examples, the polarizing filter may be configured to rotate such that the polarization angle rotation of the reflected polarized light 212 may be determined when the polarizing filter blocks the reflected light 212. In these examples, the polarization filter may be rotated until the plane of polarization of the polarization filter is “crossed” with respect to the polarization of the reflected light 212 and the polarization filter does not allow the polarized light to pass therethrough.

The detector 210 may send the detected polarization state of the polarized light 212 to the data store 106 and the processor 102 may access the detected polarization state of the polarized light 212 reflected from an eye 202 from the data store 106. The detector 210 may also be configured to detect conditions associated with the detection of the polarization state of the polarized light 212 reflected from the individual's eye 202. For instance, the detector 210 may detect a pupil dilation level of the individual's eye 202, an accommodation distance of the individual's eye 202, a gaze direction of the individual's eye 202, and an illumination level at the detector 210. The detector 210 may thus detect these conditions at a time when the polarization state of the polarized light 212 is detected. The detector 210 may also send the detected conditions at the time when the polarization state of the polarized light 212 was detected to the data store 106 for the processor 102 to access to that information.

FIG. 2B illustrates an eye tracking system 230 that is similar to the eye tracking system 200 illustrated in FIG. 2A. The eye tracking system 230 differs from the eye tracking system 200 in that the eye tracking system 230 may include a beam splitter 220 that may split the polarized light 206 directed into the eye 202 and the polarized light 212 reflected from the eye 202. In other words, the beam splitter 220 may be positioned to allow polarized light 206 from the illumination source 204 to pass through the beam splitter 220 and into the eye 202. The beam splitter 220 may also be positioned to allow polarized light 212 reflected back from the eye 202 to be reflected toward the camera 208.

With reference back to FIG. 1, the processor 102 may execute the instructions 112 to identify conditions associated with a detection of the polarization state of the light 212 reflected from the individual's eye 202. For instance, the processor 102 may identify the conditions that the detector 210 stored in the data store 106.

The processor 102 may execute the instructions 114 to determine whether the identified conditions match certain conditions with predefined difference levels. The certain conditions may be, for instance, a set of reference conditions, such as a certain pupil dilation level, a certain accommodation distance, a certain gaze direction, a certain illumination level of external light, and/or the like. In some examples, the certain conditions may be the conditions that the detector 210 detected during a previous polarized light 212 detection event, the certain conditions may be a set of conditions that may have been preselected as providing accurate glucose level measurements, the conditions that may be optimal for glucose level measurements, the conditions that optimize a signal-to-noise ratio, and/or the like. The certain conditions may be user defined, determined through testing, determined through application of artificial intelligence and/or modeling, and/or the like. In any regard, the processor 102 may set the certain conditions as the reference conditions that the processor 102 may use in determining whether the detected polarization state of the polarized light 212 is to be used to determine a glucose level of an individual.

The predefined difference levels may be user defined, based on historical data, based on modeling, and/or the like. The predefined difference levels may also be based on a desired level of precision in the glucose level determination. In other words, the predefined difference levels may be smaller for greater levels of precision and larger for lesser levels of precision. Additionally, the processor 102 may use different predefined difference levels for multiple ones of the conditions. In some examples, the predefined difference levels may be zero, in which case the processor 102 may determine whether the identified conditions are identical to the certain conditions.

The processor 102 may execute the instructions 116 to determine a variance in the detected polarization state of the polarized light 212 from a previously detected polarization state based on a determination that the identified conditions match the certain conditions within the predefined difference levels. In other words, based on the identified conditions at which the polarization state of the polarized light 212 matching the certain conditions within the predefined difference levels, the processor 102 may determine the amount of variance existing between the accessed polarization state of the light 212 reflected from the eye 202 and a previously detected polarization state of the eye 202.

Stated another way, if all of the other conditions, such as eye position or gaze, accommodation distance, pupil size, and illumination level from an external illumination source are accounted for, the remaining contributor affecting the polarization state of the detected light 212 may be the optical rotatory dispersion of the eye 202. As discussed herein, the optical rotatory dispersion (or the polarization state of the light 212) may be proportional to the glucose levels in the eye 202. The glucose levels in the eye 202 may also be equal or proportional to the individual's general blood glucose levels.

In some examples, the processor 102 may determine a glucose level of the individual based on the detected polarization state of the individual's eye 202. The processor 102 may determine the glucose level based on predefined correlations between polarization states and glucose levels. The processor 102 may also compare the determined glucose level with the glucose level corresponding to the previously detected polarization state of the individual's eye 202. The processor 102 may further execute the instructions 118 to determine how the glucose level of the individual has changed over time based on the comparison.

The processor 102 may also execute the instructions 120 to, based on a determination that the identified conditions do not match the certain conditions within the predefined difference levels, disregard and/or discard the detected polarization state of the reflected light 212.

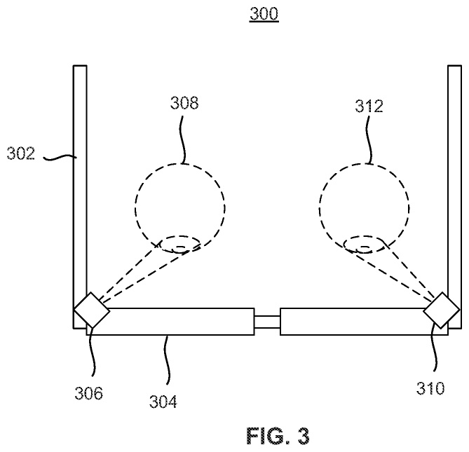

In some examples, the eye tracking system 200, 230 may be incorporated in a head-mounted device, such as a head-mounted AR and/or VR system. FIG. 3 illustrates a top view of a head-mounted device 300 in which either of the eye tracking systems 200, 230 depicted in FIGS. 2A, 2B may be incorporated, according to an example. The head-mounted device 300 may be a wearable eyewear or a near-eye display, in the form of a pair of smartglasses, glasses, or other similar eyewear, according to an example. In some examples, the head-mounted device 300 may be configured to operate as a virtual reality display, an augmented reality display, and/or a mixed reality display. In some examples, the head-mounted device 300 may be eyewear, in which a user of the head-mounted device 300 may see through lenses in the head-mounted device 300.

In some examples, the head-mounted device 300 may include a frame 302 and a display 304. In some examples, the display 304 may be configured to present media or other content to a user. In some examples, the display 304 may include display electronics and/or display optics. For example, the display 304 may include a liquid crystal display (LCD) display panel, a light-emitting diode (LED) display panel, or an optical display panel (e.g., a waveguide display assembly). In some examples, the display 304 may also include any number of optical components, such as waveguides, gratings, lenses, mirrors, etc. In other examples, the display 304 may be omitted and instead, the head-mounted device 300 may include lenses that are transparent and/or tinted, such as sunglasses.

In some examples, the head-mounted device 300 may include any number of depth sensors, motion sensors, position sensors, inertial sensors, and/or ambient light sensors. In some examples, the various sensors may include any number of image sensors configured to generate image data representing different fields of views in one or more different directions. In some examples, the various sensors may be used as input devices to control or influence the displayed content of the head-mounted device 300, and/or to provide an interactive virtual reality (VR), augmented reality (AR), and/or mixed reality (MR) experience to a user of the head-mounted device 300. In some examples, the various sensors may also be used for stereoscopic imaging or other similar application.

As also shown, an eye tracking system 306, which may include the same features as either of the eye tracking systems 200, 230, may be included in the frame 302 of the head-mounted device 300. For instance, the eye tracking system 306 may be mounted on the frame 302, for instance, close to the display 304. In some examples, the illumination source 204 and the camera 208 of the eye tracking system 306 may be pointed directly at a user's eye 308 as shown in FIG. 3. In other examples, the display 304 may include optical components, such as waveguides, to direct light from the illumination source 204 through the display 304 and into the user's eye 308 and to direct light reflected from the user's eye 308 to the camera 208. In addition, in some examples, the head-mounted device 300 may include a second eye tracking system 310 positioned to track a user's second eye 312. The second eye tracking system 310 may operate similarly to the eye tracking system 306.

The eye tracking systems 306, 310 may be employed in the head-mounted device 300 to track various conditions of a user's eye or eyes 308, 312. For instance, the eye tracking systems 306, 310 may track accommodation distances of the user's eyes 308, 312, the gaze directions of the user's eyes 308, 312, the pupil dilations etc., and may use the tracked information to display images to a user. By way of example, the tracked information may be used to determine locations on the display 304 of the head-mounted device 300 at which various items are displayed to a user.

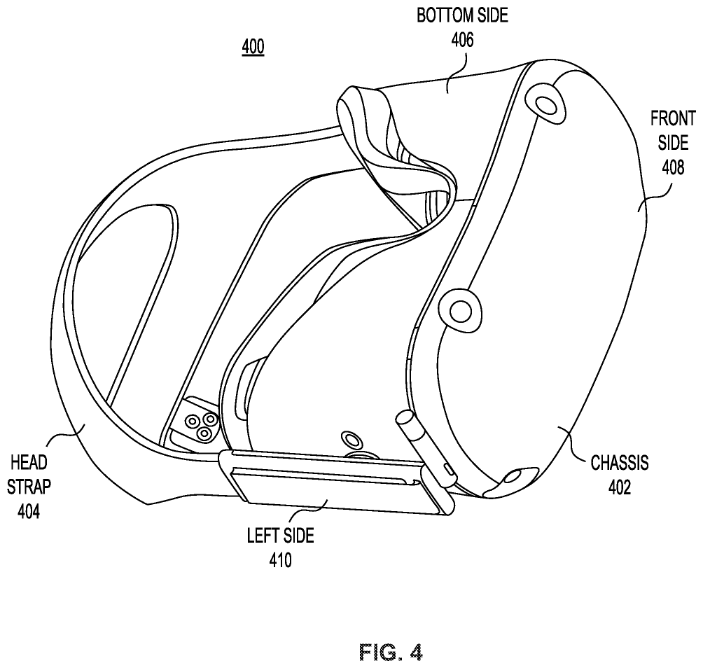

FIG. 4 illustrates a perspective view of a head-mounted device 400, such as a near-eye display device, that may include either of the eye tracking systems depicted in FIGS. 2A, 2B, according to an example. The head-mounted device 400 may include either of the eye tracking systems 200, 230 depicted in FIGS. 2A and 2B. Particularly, the head-mounted device 400 may include one or more eye tracking systems 200, 230 positioned inside of the head-mounted device 400 and may be positioned to track one or both of a user's eyes as discussed herein.

In some examples, the head-mounted device 400 may be part of a virtual reality (VR) system, an augmented reality (AR) system, a mixed reality (MR) system, another system that uses displays or wearables, or any combination thereof. In some examples, the head-mounted device 400 may include a chassis 402 and a head strap 404. FIG. 4 shows a bottom side 406, a front side 408, and a left side 410 of the chassis 402 in the perspective view. In some examples, the head strap 404 may have an adjustable or extendible length. In particular, in some examples, there may be a sufficient space between the chassis 402 and the head strap 404 of the head-mounted device 400 for allowing a user to mount the head-mounted device 400 onto the user's head. In some examples, the head-mounted device 400 may include additional, fewer, and/or different components.

In some examples, the head-mounted device 400 may present, to a user, media or other digital content including virtual and/or augmented views of a physical, real-world environment with computer-generated elements. Examples of the media or digital content presented by the head-mounted device 400 may include images (e.g., two-dimensional (2D) or three-dimensional (3D) images), videos (e.g., 2D or 3D videos), audio, or any combination thereof. In some examples, the images and videos may be presented to each eye of a user by one or more display assemblies (not shown in FIG. 4) enclosed in the chassis 402 of the head-mounted device 400.

In some examples, the apparatus 100 may include the eye tracking system 200. In these examples, the apparatus 100 may be one of the head-mounted devices 300, 400. In these examples, the apparatus 100 may include the eye tracking system 200, 230 discussed herein. In other words, the apparatus 100 may include an illumination source 204 and a camera 208 as discussed herein. In addition, the processor 102 (and in some examples, the memory 104) may be housed within or on the head-mounted devices 300, 400.

According to examples, the eye tracking system 200, 230 may detect a polarization state of the reflected polarized light 212 multiple times when the user is using the apparatus 100, e.g., the head-mounted device 300, 400. That is, for instance, the illumination source 204 may output light at a known polarization state into a user's eye 202 and the detector 210 may detect a polarization state of light reflected from the eye 202 at certain times while the user is using the apparatus 100. For instance, the eye tracking system 200, 230 may detect the polarization state of the reflected polarized light 212 at certain intervals of time, e.g., once a minute, once every 15 minutes, once every 30 minutes, or the like. The eye tracking system 200, 230 may also send the detected polarization states to the data store 106. The eye tracking system 200, 230 may further track the conditions associated with the detections of the polarization states and may send information corresponding to the tracked conditions to the data store 106.

According to examples, the processor 102 may access the multiple detected polarization states of the light 212 reflected from the user's eye 202. In addition, for each of the accessed multiple detected polarization states, the processor 102 may identify conditions associated with the detection of the detected polarization state and may determine whether the identified conditions match the certain conditions within the predefined difference levels as discussed herein. The processor 102 may also determine a variance in the detected polarization state and a previously detected polarization state based on a determination that the identified conditions match the certain conditions with the predefined difference levels.

The processor 102 may further determine the change in the glucose level of the individual based on the determined variance of the detected polarization states of the light from the previously detected polarization state. Additionally, the processor 102 may track the changes in the glucose level of the individual over time and may store those changes in the data store 106. The processor 102 may also present the determined changes in glucose levels to the user, for instance, via a display of the apparatus 100.

However, for the accessed multiple detected polarization states that were detected under conditions that do not match the certain conditions within the predefined difference levels, the processor 102 may disregard and/or discard the detected polarization states. The processor 102 may disregard these detected polarization states because the changes in the polarization states may have occurred due to changes in one or more of the conditions in addition to or other than a change in the glucose level in the user's eye 202.

In some instances, environmental light may potentially corrupt the polarization state measurements by adding noise to the signal, e.g., the reflected light 212 captured by the camera 208. This type of noise may be more prevalent when the head-mounted device 300 depicted in FIG. 3 is employed to detect the polarization state of the light reflected from an individual's eye 202. To compensate for this possibility, the illumination source 204 may be employed to output light at multiple wavelengths, e.g., 850 nm and 890 nm. The changes in the detected polarization states resulting from the different wavelengths of light may be used to decouple the light that is emitted from the illumination source 204 and the environment light. In other words, by changing the wavelength of the light from the illumination source 204, the noise that is related to an environmental light may be changed.

For instance, wavelengths may rapidly be changed and the changes in polarization states of the reflected light 212 resulting from the changes in the wavelengths may be detected. The processor 102 may determine that there may be a significant amount of interference from the environment light in instances in which the detected changes in the polarization states are different from each other. The processor 102 may disregard or discard those measurements. However, the processor 102 may determine that there may not be a significant amount of interference from the environment light in instances in which the detected changes in the polarization states are similar to each other. The processor 102 may determine that those detected changes in the polarization states may be used to determine the glucose level.

In other examples, the illumination source 204 may be a switchable multiband illumination source and the camera 208 may be a multispectral polarization sensitive camera. In these examples, the camera 208 may concurrently detect changes in polarization states for light outputted at multiple wavelengths from the illumination source 204. The processor 102 may also disregard or discard the detected changes in polarization states that have been found to differ significantly from each other.

In some examples, the illumination source 204 may be a laser, VCSEL, or other narrowband illumination source, that may be coupled with a beam-scanning system. The beam-scanning system may be a micro-electromechanical system (MEMS) based or other type of scanner. In addition, the camera 208 may include a detector 210 that may capture light that is sufficient for calculating the angle of polarization at a single point. For instance, the camera 208 may include detectors 210 with wire grid polarization filters (e.g., filters that polarize different angles of light such as 0°, 45°, 90°, and 135°. The processor 102 may determine whether to use or discard the detected changes in polarization states based on whether the detected changes in polarization states by the detectors 210 differ significantly with respect to each other. A determination as to whether the differences are significant may be based on historical data, user-defined variances, and/or the like.

In some examples in which a separate eye tracking system 306, 310 is employed to track both eyes 308, 312 of a user, the processor 102 may determine whether the changes in the polarization states detected by the eye tracking systems 306, 310 differ significantly from each other. For instance, each of the eye tracking systems 306, 310 may deliver light at a different wavelength to the respective eyes 308, 312. If the polarization states differ significantly from each other, the processor 102 may disregard or discard those detected polarization states.

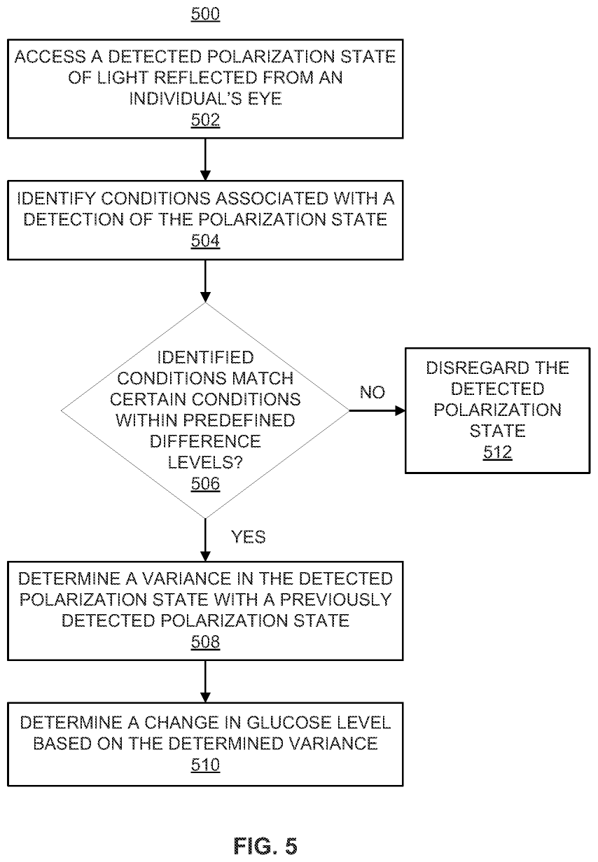

Various manners in which the processor 102 of the apparatus 100 may operate are discussed in greater detail with respect to the method 500 depicted in FIG. 5. FIG. 5 illustrates a flow diagram of a method 500 for determining a variance in a detected polarization state of light over a previously detected polarization state of light reflected from an individual's eye, according to an example. It should be understood that the method 500 depicted in FIG. 5 may include additional operations and that some of the operations described therein may be removed and/or modified without departing from the scope of the method 500. The description of the method 500 is made with reference to the features depicted in FIGS. 1-4 for purposes of illustration.

At block 502, the processor 102 may access a detected polarization state of light reflected from an individual's eye 202.

At block 504, the processor 102 may to identify conditions associated with a detection of the polarization state of the light 212 reflected from the individual's eye 202. The conditions may include one or more of, at a time when the polarization state of the light was detected a pupil dilation level of the individual's eye 202, an accommodation distance of the individual's eye 202, a gaze direction of the individual's eye 202, and an illumination level at a detector 210 of the polarization state of the light caused by an external illumination source.

At block 506, the processor 102 may determine whether the identified conditions match certain conditions within predefined difference levels.

At block 508, the processor 102 may determine a variance in the detected polarization state of the polarized light 212 from a previously detected polarization state based on a determination that the identified conditions match the certain conditions within the predefined difference levels.

At block 510, the processor 102 may determine how the glucose level of the individual has changed over time based on the comparison.

At block 512, the processor 102 may, based on a determination that the identified conditions do not match the certain conditions within the predefined difference levels, disregard the detected polarization state of the light 212.

Some or all of the operations set forth in the method 500 may be included as a utility, program, or subprogram, in any desired computer accessible medium. In addition, the method 500 may be embodied by a computer program, which may exist in a variety of forms both active and inactive. For example, they may exist as machine-readable instructions, including source code, object code, executable code or other formats. Any of the above may be embodied on a non-transitory computer readable storage medium.

Examples of non-transitory computer readable storage media include computer system RAM, ROM, EPROM, EEPROM, and magnetic or optical disks or tapes. It is therefore to be understood that any electronic device capable of executing the above-described functions may perform those functions enumerated above.

In the foregoing description, various inventive examples are described, including devices, systems, methods, and the like. For the purposes of explanation, specific details are set forth in order to provide a thorough understanding of examples of the disclosure. However, it will be apparent that various examples may be practiced without these specific details. For example, devices, systems, structures, assemblies, methods, and other components may be shown as components in block diagram form in order not to obscure the examples in unnecessary detail. In other instances, well-known devices, processes, systems, structures, and techniques may be shown without necessary detail in order to avoid obscuring the examples.

The figures and description are not intended to be restrictive. The terms and expressions that have been employed in this disclosure are used as terms of description and not of limitation, and there is no intention in the use of such terms and expressions of excluding any equivalents of the features shown and described or portions thereof. The word “example” is used herein to mean “serving as an example, instance, or illustration.” Any embodiment or design described herein as “example’ is not necessarily to be construed as preferred or advantageous over other embodiments or designs.

Although the methods and systems as described herein may be directed mainly to digital content, such as videos or interactive media, it should be appreciated that the methods and systems as described herein may be used for other types of content or scenarios as well. Other applications or uses of the methods and systems as described herein may also include social networking, marketing, content-based recommendation engines, and/or other types of knowledge or data-driven systems.

1. An apparatus, comprising:
a processor; and
a memory on which is stored machine-readable instructions that when executed by the processor, cause the processor to:
access a detected polarization state of light reflected from an individual's eye;
identify conditions associated with a detection of the polarization state of the light reflected from the individual's eye;
determine whether the identified conditions match certain conditions within predefined difference levels; and
determine a variance in the detected polarization state of the light from a previously detected polarization state based on a determination that the identified conditions match the certain conditions within the predefined difference levels, wherein a change in a glucose level of the individual is to be determined based on the determined variance.

2. The apparatus of claim 1, wherein the conditions comprise one or more of, at a time when the polarization state of the light was detected:
pupil dilation level of the individual's eye;
accommodation distance of the individual's eye;
gaze direction of the individual's eye; and
an illumination level at a detector of the polarization state of the light caused by an external illumination source.

3. The apparatus of claim 1, wherein the instructions cause the processor to:
disregard the detected polarization state of the light based on a determination that the identified conditions do not match the certain conditions within the predefined difference levels.

4. The apparatus of claim 1, further comprising:
an eye tracking system including:
a polarization sensitive detector; and
an illumination source to direct light at a known polarization state into the individual's eye, wherein the processor is to access the polarization state of light detected by the polarization sensitive detector in the eye tracking system.

5. The apparatus of claim 4, wherein eye tracking system is to detect one or more of, at a time when the polarization state of the light is detected:
a pupil dilation level of the individual's eye;
an accommodation distance of the individual's eye;
a gaze direction of the individual's eye; and
an illumination level at the polarization sensitive detector caused by an external illumination source.

6. The apparatus of claim 4, wherein the apparatus comprises a head-mounted device and wherein the individual is a user of the head-mounted device.

7. The apparatus of claim 6, wherein the eye tracking system is to detect a polarization state of light multiple times when the individual is using the apparatus and wherein the instructions cause the processor to:
access multiple detected polarization states of the light reflected from the individual's eye; and
for each of the accessed multiple detected polarization states,
identify conditions associated with the detection of the detected polarization state;
determine whether the identified conditions match the certain conditions within the predefined difference levels; and
determine a variance in the detected polarization state and a previously detected polarization state based on a determination that the identified conditions match the certain conditions with the predefined difference levels.

8. The apparatus of claim 7, wherein the instructions cause the processor to:
for the accessed multiple detected polarization states that were detected under conditions that do not match the certain conditions within the predefined difference levels, disregard the detected polarization states.

9. The apparatus of claim 1, wherein the instructions cause the processor to:
determine the change in the glucose level of the individual based on the determined variance of the detected polarization state of the light from the previously detected polarization state.

10. A head-mounted device comprising:
a frame to be worn by an individual;
a processor housed in or on the frame; and
an eye tracking system mounted on the frame, the eye tracking system including:
an illumination source to direct light at a known polarization state into the individual's eye, wherein light is to be reflected from the individual's eye at a reflected polarization state; and
a polarization sensitive detector to detect the reflected polarization state of the light reflected from the individual's eye; and
wherein the processor is to:
access the reflected polarization state of the light;
identify conditions associated with the detection of the reflected polarization state of the light;
determine whether the identified conditions match certain conditions within predefined difference levels; and
determine a variance in the detected polarization state of the light from a previously detected polarization state based on a determination that the identified conditions match the certain conditions within the predefined difference levels, wherein a change in a glucose level of the individual is to be determined based on the determined variance.

11. The head-mounted device of claim 10, wherein the conditions comprise one or more of, at a time when the polarization state of the light was detected:
pupil dilation level of the individual's eye;
accommodation distance of the individual's eye;
gaze direction of the individual's eye; and
an illumination level at a detector of the polarization state of the light caused by an external illumination source.

12. The head-mounted device of claim 10, wherein the processor is to:
disregard the detected polarization state of the light based on a determination that the identified conditions do not match the certain conditions within the predefined difference levels.

13. The head-mounted device of claim 10, wherein eye tracking system is to detect one or more of, at a time when the polarization state of the light is detected:
a pupil dilation level of the individual's eye;
an accommodation distance of the individual's eye;
a gaze direction of the individual's eye; and
an illumination level at the polarization sensitive detector caused by an external illumination source.

14. The head-mounted device of claim 10, wherein the eye tracking system is to detect a polarization state of light multiple times when the individual is using the head-mounted device and wherein the processor is to:
access multiple detected polarization states of the light reflected from the individual's eye; and
for each of the accessed multiple detected polarization states,
identify conditions associated with the detection of the detected polarization state;
determine whether the identified conditions match the certain conditions within the predefined difference levels; and
determine a variance in the detected polarization state and a previously detected polarization state based on a determination that the identified conditions match the certain conditions with the predefined difference levels.

15. The head-mounted device of claim 14, wherein the processor is to:
for the accessed multiple detected polarization states that were detected under conditions that do not match the certain conditions within the predefined difference levels, disregard the detected polarization states.

16. The head-mounted device of claim 10, wherein the processor is to:
determine the change in the glucose level of the individual based on the determined variance of the detected polarization state of the light from the previously detected polarization state.

17. A method of determining a variance in a detected polarization state of light, the method comprising:
accessing, by a processor, a detected polarization state of light reflected from an individual's eye;
identifying, by the processor, conditions associated with a detection of the polarization state of the light reflected from the individual's eye;
determining, by the processor, whether the identified conditions match certain conditions within predefined difference levels; and
determining, by the processor, a variance in the detected polarization state of the light from a previously detected polarization state based on a determination that the identified conditions match the certain conditions within the predefined difference levels.

18. The method of claim 17, further comprising:
determining a change in a glucose level of the individual based on the determined variance of the detected polarization state of the light from the previously detected polarization state.

19. The method of claim 17, further comprising:
disregarding the detected polarization state of the light based on a determination that the identified conditions do not match the certain conditions within the predefined difference levels.

20. The method of claim 17, further comprising:
accessing multiple detected polarization states of the light reflected from the individual's eye; and
for each of the accessed multiple detected polarization states,
identifying conditions associated with the detection of the detected polarization state;
determining whether the identified conditions match the certain conditions within the predefined difference levels; and
determining a variance in the detected polarization state and a previously detected polarization state based on a determination that the identified conditions match the certain conditions with the predefined difference levels.
